(19) United States
(12) Patent Application Publication
(10) Pub. No.: US 2015/0275187 A1
(43) Pub. Date: Oct. 1, 2015

(54) FURFURAL-RESISTANT GENE AND FURFURAL-RESISTANT STRAINS COMPRISING THE SAME

(71) Applicant: KOREA INSTITUTE OF SCIENCE AND TECHNOLOGY, Seoul (KR)

(72) Inventors: Han Min WOO, Seoul (KR); Hong-Sil PARK, Gwangju (KR); Gyeong Taek Gong, Seoul (KR); Youngsoon UM, Seoul (KR); Yunje KIM, Seoul (KR); Dong Jin SUH, Seoul (KR)

(73) Assignee: KOREA INSTITUTE OF SCIENCE AND TECHNOLOGY, Seoul (KR)

(21) Appl. No.: 14/338,770

(22) Filed: Jul. 23, 2014

(30) Foreign Application Priority Data
Mar. 28, 2014 (KR) 10-2014-0036970

(51) Int. Cl. C12N 9/02; C12Q 1/68; C12P 13/00

(52) U.S. Cl. CPC C12N 9/001; C12P 13/001; C12Q 1/6876; C12Y 103/01032; C12Q 2600/16; C12Q 2600/158

(57) ABSTRACT

The furfural-resistant strain containing the furfural-resistant gene according to the present disclosure may be effectively grown in a furfural-containing medium. Accordingly, the problem that microorganism fermentation was difficult because toxic by-products such as furfural are contained in a hydrolysate derived from inedible lignocellulosic biomass may be solved. Further, according to the method for producing a strain of the present disclosure, the resistant gene may be selected from relatively small number of target genes. Thus, time, cost and the like for developing the resistant strain may be saved. Further, this method for identifying genes may be broadly applied to methods for identifying various unknown functional genes in addition to the furfural-resistant gene.

### CROSS-REFERENCE TO RELATED APPLICATION

This application claims priority to Korean Patent Application No. 10-2014-0036970, filed on Mar. 28, 2014, and all the benefits accruing therefrom under 35 U.S.C. §119, the contents of which in its entirety are herein incorporated by reference.

### BACKGROUND

1. Field

Disclosed herein is a gene having resistance to furfural, which is a by-product of a hydrolysate derived from lignocellulosic biomass, and a strain comprising the same.

Description about National Support Research and Development

This study is conducted by the support of Korea Ministry of Science, ICT and Future Planning under the supervision of Korea Institute of Science and Technology, and research title is development of technique for manufacturing next generation fuel/material by integrated utilization of lignocellulosic biomass (2N36860) (Research management agency: Korea Research Council of Fundamental Science and, Grant Number: CAP-11-1: Creative Allied Program[CAP]).

2. Description of the Related Art

As concerns about global warming and oil resource depletion rise, world bio research fields pay more attention to development of alternative energy sources and alternative materials of petrochemicals. For this, studies into production of bio-fuels and platform chemicals from lignocellulosic biomass by fermentation of microorganism strain are actively going on. The lignocellulosic biomass mainly consists of cellulose, hemicellulose, lignin and the like, and can be converted to renewable fuels, plastics and other various chemicals by fermentation of microorganism.

In order to use the lignocellulosic biomass to microorganism fermentation, a pre-treatment process for hydrolyzing cellulose to sugar is needed. However, various by-products, which are made during the pre-treatment process, as well as sugars such as glucose and xylose, which can be used by microorganisms, are contained in a hydrolysate, which is made during the pre-treatment for sugar hydrolysis using acid at high temperature and pressure. Examples of the by-products made during the pre-treatment process for sugar hydrolysis are furfural, hydroxymethylfurfural (HMF), acetic acid and the like. Because these have toxicity to microorganisms, there is a problem of decreased productivity on the fermentation process using the hydrolysate. Accordingly, development of a strain having resistance to a toxic material in the hydrolysate is necessary.

Furfural made from xylose, among the by-product existing in the hydrolysate, is one of important materials inhibiting microorganism fermentation in the hydrolysate, and the toxicity of the hydrolysate has considerable relevance to the concentration of the furfural. The furfural is converted to furfuryl alcohol, which is relatively less toxic than the furfural in cells. In the process, it uses NADH or NADPH, and intracellular amount of the NAD(P)H becomes unbalanced, thereby inhibiting cell growth and fermentation. In addition, it is known that its toxicity becomes severe because it causes genetic mutation, makes cell membrane weak, and interacts with other materials in the hydrolysate such as hydroxymethylfurfural and acetic acid.

### REFERENCES OF THE RELATED ART

### Patent Document

International Patent Publication No. WO2012-135420A2 (2012 Oct. 4)
Korean Patent Publication No. 10-2003-0040605 (2003 May 23)

### Non-Patent Document

Wang, X., E. Miller, et al., 2011, “Increased furfural tolerance due to overexpression of NADH-dependent oxidoreductase FucO in Escherichia coli strains engineered for the production of ethanol and lactate.” Applied and environmental microbiology 77(15): 5132-5140
Sasano, Y., D. Watanabe, et al., 2012, “Overexpression of the yeast transcription activator Msn2 confers furfural resistance and increases the initial fermentation rate in ethanol production.” Journal of bioscience and bioengineering 113(4): 451-455

### SUMMARY

The present disclosure is directed to providing a gene having resistance to furfural, which is a toxic by-product contained in a hydrolysate derived from lignocellulosic biomass, and a strain containing the same.

In one aspect, there is provided a furfural-resistant gene containing at least one genetic sequence selected from the group consisting of the cg1661 (SEQ ID No.: 1) and cg1310 (SEQ ID No.: 2) gene, and a furfural-resistant strain containing the same.

In another aspect, there is provided a screening method of the furfural-resistant gene, which contains: selecting a target gene for identifying the furfural-resistant gene; and after inserting the target gene into a wild type and overexpressing thereof, selecting a gene showing furfural resistance as the furfural-resistant gene.

The furfural-resistant strain containing the furfural-resistant gene according to the present disclosure shows faster growth speed than the wild type (wild type strain) in the furfural-containing medium. Thus, it may increase production rate of the material, which is produced by the wild type. Accordingly, the problem that microorganism fermentation was difficult because toxic by-products such as furfural are contained in a hydrolysate derived from inedible lignocellulosic biomass may be solved. Thus, it may effectively produce food additives, feed additives and the like such as amino acids from inedible biomass as well as the existing starch-based biomass without growth inhibition or production rate reduction by the toxic materials in a hydrolysate.

Further, according to the method for producing a strain of the present disclosure, the resistant gene may be selected from relatively small number of target genes. Thus, time, cost and the like for developing the resistant strain may be saved. Further, this method for identifying genes may be broadly applied to methods for identifying various unknown functional genes in addition to the furfural-resistant gene.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 is a graph showing growth curve of Corynebacterium glutamicum wild type strain (wild type) in a medium containing furfural of various concentrations (0, 6.5, 13 and 20 mM);

FIGS. 2a to 2c are graphs showing the result of analyzing the concentrations of furfural and furfuryl alcohol in the furfural-containing medium according to the growth of Corynebacterium glutamicum, wherein FIG. 2a is the case of the medium containing 6.5 mM furfural, FIG. 2b is the case of the medium containing 13 Mm furfural, and FIG. 2c is the case of the medium containing 20 mM furfural;

FIG. 3 is an image schematically showing the process of homology search of FucO in Corynebacterium glutamicum;

FIG. 4 is a graph comparing relative growth of target gene-transformed strains (pAN6-cg0310, pAN6-cg1310, pAN6-cg1661 and pAN6-cg3374) and a positive control group (pAN6-b2799) in the medium containing 6.5 mM furfural, with growth rate of a negative control group (pAN6) in the medium not containing the furfural, respectively; and

FIG. 5 is a graph comparing relative growth of target gene-transformed strains (pAN6-cg0310, pAN6-cg1310, pAN6-cg1661 and pAN6-cg3374) and a positive control group (pAN6-b2799) in the medium containing 13 mM furfural, with growth rate of a negative control group (pAN6) in the medium not containing the furfural, respectively.

### DETAILED DESCRIPTION

Exemplary embodiments now will be described more fully hereinafter with reference to the accompanying drawings, in which exemplary embodiments are shown.

Embodiments of the present disclosure provide a furfural-resistant gene containing at least one genetic sequence selected from the group consisting of cg1661 (SEQ ID No.: 1) and cg1310 (SEQ ID No.: 2) gene.

The furfural is an organic compound made from by-products during sugar hydrolysis process of various biomass, and has a chemical formula of OC₄H₃CHO and a chemical structure of the following formula (I). The furfural is toxic, and therefore, it deteriorates production speed of microorganism and production rate of fermentation product when fermenting microorganism in a hydrolysate of biomass.

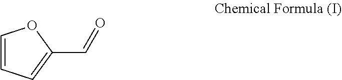

Accordingly, embodiment of the present disclosure provides a furfural-resistant strain, which contains the furfural-resistant gene, thereby having excellent growth capability under furfural-containing environment. Specifically, the furfural-resistant strain according to embodiments of the present disclosure is a strain, wherein the furfural-resistant gene containing the cg1661 (SEQ ID No.: 1) and cg1310 (SEQ ID No.: 2) gene is inserted in a wild type. In one embodiment, the wild type is Corynebacterium glutamicum, specifically Corynebacterium glutamicum ATCC 13032 (ACCESSION NC_{—}006958, VERSION NC_{—}006958.1 GI:62388892). The insertion of the gene is conducted by inserting the furfural-resistant gene into the wild type using a vector and then overexpressing thereof, and the vector may be, for example, a plasmid such as pAN6. In one embodiment, the furfural-resistant strain is Corynebacterium glutamicum ATCC 13032/pAN6-cg1661 (KCTC 12565BP).

The furfural-resistant strain according to embodiments of the present disclosure may be grown in a furfural-containing medium. Further, growth of the furfural-resistant strain according to one embodiment in the furfural-containing medium is increased about 1.4 folds or more, more specifically about 1.8 folds or more, compared to a wild type. When the wild type is Corynebacterium glutamicum, the Corynebacterium glutamicum wild type (wild type strain) is hard to be grown in the hydrolysate derived from lignocellulosic biomass due to toxicity of the furfural contained in the hydrolysate. However, the furfural-resistant strain according to embodiments of the present disclosure has furfural resistance by containing the furfural-resistant gene of the present disclosure. Thus, it may be effectively grown in the hydrolysate derived from lignocellulosic biomass, thereby having further increased amino acid productivity, compared to the wild type.

Accordingly, embodiments of the present disclosure may provide a method for producing amino acids, which contains inoculating the furfural-resistant strain into the hydrolysate derived from lignocellulosic biomass.

Further, another embodiment of the present disclosure may provide a method for screening the furfural-resistant gene, which contains:

selecting a target gene for identifying the furfural-resistant gene; and

after inserting the target gene into the wild type and overexpressing thereof, selecting a gene showing furfural resistance as the furfural-resistant gene.

In order to select the target gene for identifying the furfural-resistant gene, embodiments of the present disclosure may contains at least one of:

searching genes, which are expected to have furfural-resistance through literature search, and selecting a gene, which is similar with the gene searched through the literature, among genes of the wild type, as the target gene; and

analyzing gene expression pattern depending on furfural stress using microarray, and selecting a gene showing high expression level as the target gene.

In one embodiment, selecting the target gene through literature search may contain searching furfural-resistant genes known in literature, and selecting genes showing high similarity in the wild type by homology search as the target gene. For example, from [Wang, X., E. Miller, et al., 2011, “Increased furfural tolerance due to overexpression of NADH-dependent oxidoreductase FucO in Escherichia coli strains engineered for the production of ethanol and lactate.” Applied and environmental microbiology 77(15): 5132-5140], it may be found that the NADH-dependent oxidoreductase (fucO) converts the furfural to furfuryl alcohol (see the following chemical formula (II)). Then, the genes in the wild type, which are similar with the searched FucO, may be selected as the target gene. At this time, the contents of the literature in its entirety are herein incorporated by reference.

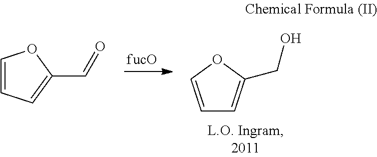 [SMILES: O=Cc1ccco1 ; OCc1ccco1]

In one embodiment, when the parent strain is Corynebacterium glutamicum, cg1310 (rolM, maleylacetate reductase, SEQ ID No.: 2), which is similar with the genes searched by the literature in Corynebacterium glutamicum genes, may be selected as the target gene.

In another embodiment, specifically, selecting the target gene using microarray may contain: treating the furfural to the wild type at different concentrations, conducting microarray to mRNA of the wild type, and then selecting genes, whose gene expression change depending on furfural stress, namely, whose expression pattern increased by the furfural, is judged to have relevance to furfural resistance, as the target gene. For example, when using Corynebacterium glutamicum as the wild type, cg0310 (SEQ ID No.: 3), cg3374 (SEQ ID No.: 4), cg1661 (SEQ ID No.: 1) and cg3399 (SEQ ID No.: 5) may be selected as the target gene.

In embodiments of the present disclosure, a gene having high resistance to the furfural may be finally selected as the furfural-resistant gene, by inserting the selected target gene into the wild types and overexpressing thereof. Specifically, the selected target genes are cloned and inserted into the wild types to obtain target gene-overexpressed strains, and then these strains are cultured in the furfural-containing medium. Then, their growth speed is compared with growth speed of strains with an expression vector (e.g.: pAN6), in which the target gene is not inserted, and the target genes, which are inserted in the strains showing increased growth speed, may be selected as the furfural-resistant genes. For example, when using Corynebacterium glutamicum as the wild type, the target genes cg1310, cg0310, cg3374, cg1661 and cg3399 are genetically recombined and inserted into the wild type, respectively, and overexpressed. Then, a recombinant strain, which substantially shows higher cell growth rate than the wild type, is selected, and the target gene inserted in the recombinant strain is finally selected as the furfural-resistant genes. In one embodiment, the final furfural-resistant gene selected in the above step may contain at least one gene of membrane protein (efflux permease)-type cg1661 and reductase-type cg1310.

The examples (and experiments) will now be described. The following examples (and experiments) are for illustrative purposes only and not intended to limit the scope of the present disclosure.

### Example 1

### Confirm of Growth Inhibitory Effect of Furfural in Wild type

In order to develop a furfural-resistant strain, first of all, Corynebacterium glutamicum is selected as a wild type, and then, growth inhibitory effect of furfural at Corynebacterium glutamicum wild type strain (wild type), which is Corynebacterium glutamicum ATCC 13032, depending on concentration, and concentrations of furfural and furfuryl alcohol in a culture solution depending on cell growth are confirmed.

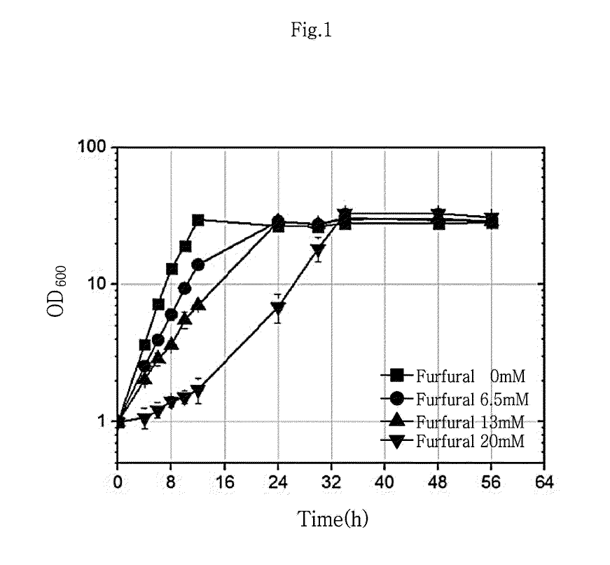

Specifically, as the culture solution, 2% glucose as a carbon source is added to a minimal medium, CGXII medium, and furfural of 6.5 mM, 13 mM and 20 mM are added thereto, respectively, to compare growth speed with the case not adding the furfural. At this time, the composition of the CGXII medium is 20 g/L (NH₄)₂50₄, 5 g/L Urea, 1 g/L KH₂PO₄, 1 g/L K₂HPO₄, 0.25 g/L MgSO₄.7H₂O, 42 g/L MOPS, 10 mg/L CaCl₂, 0.2 mg/L Biotin, 0.03 g/L Protecatechuic acid, Trace metal (10 mg/L FeSO₄.7H₂O, 10 mg/L MnSO₄.H₂O, 1 mg/L ZnSO₄.7H₂O, 0.2 mg/L CuSO₄.7H₂O, 0.02 mg/L NiCl₂.6H₂O). Culture is conducted at 30° C. after inoculating the cells (initial OD₆₀₀=1) into the culture solution 50 mL in a 250 mL Erlenmeyer flask. As a result of the culture, as shown in FIG. 1, it is confirmed that the growth speed of the strain becomes slow as the furfural concentration in the medium is increased, and the growth is reduced at about 50% or more even at the lowest concentration of 6.5 mM, compared with the medium not containing the furfural.

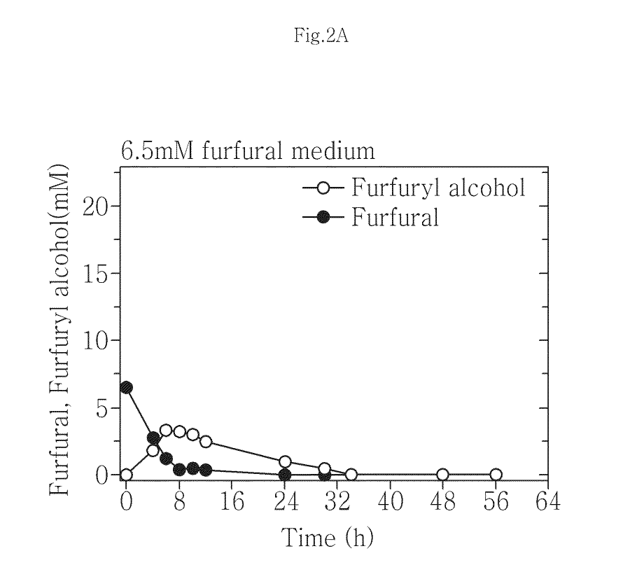

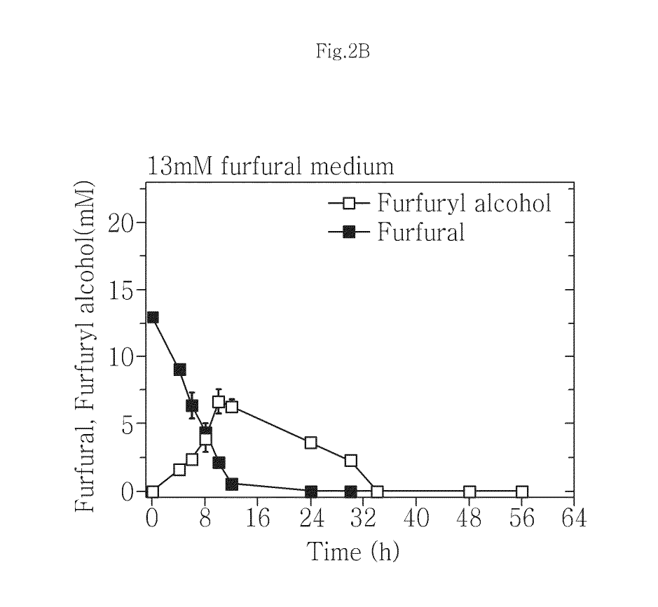

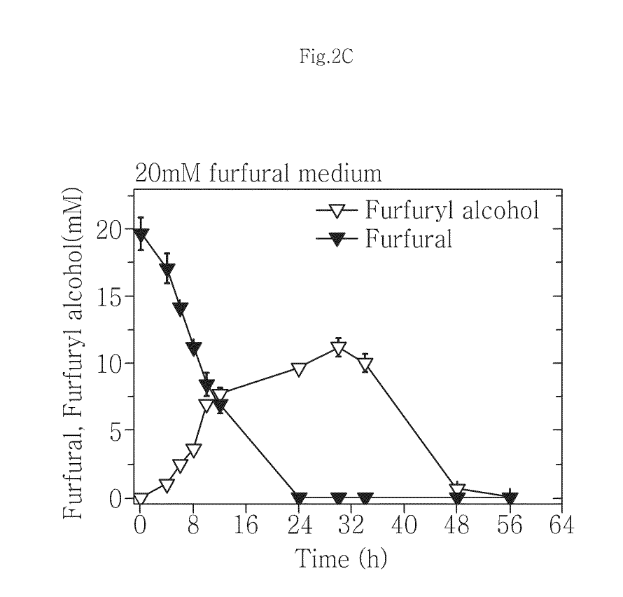

Further, in order to check the concentration changes of the furfural and furfuryl alcohol in the medium according to the growth of Corynebacterium glutamicum in the medium containing 6.5 mM, 13 mM and 20 mM furfural by the culture time, respectively, the culture solution is analyzed using gas chromatography (GC). At this time, the culture solutions obtained by each collection time are centrifuged at 12,000 rpm for 5 min, and the supernatants are filtered and used for the analysis. The results of the culture solution analysis are shown in FIG. 2a, FIG. 2b and FIG. 2c. As shown in FIGS. 2 a, b and c, it is confirmed that entire furfural added to the medium is introduced into the cells regardless of the concentration and converted to furfuryl alcohol.

### Example 2

### Selection of Target Gene for identifying Furfural-Resistant Gene

In order to select a target gene for identifying the furfural-resistant gene, two methods, i.e., a method of searching literatures and a method using microarray are used.

Method of Searching Literatures

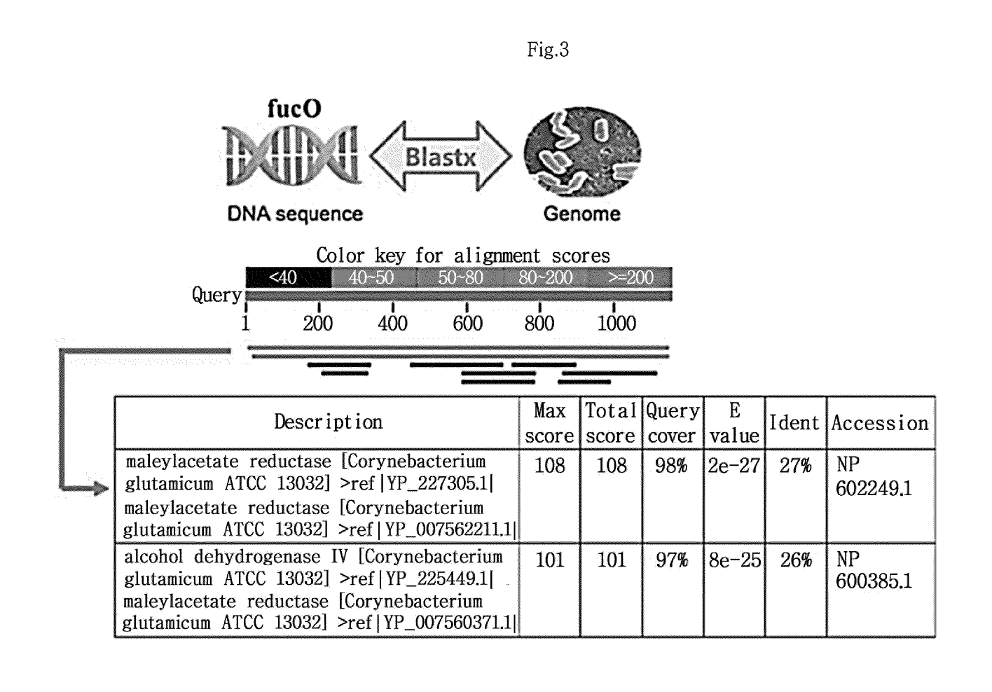

Because it is confirmed that the furfural added in the medium is converted to the furfuryl alcohol in the cells in Example 1, literature search is conducted to find a furfural reductase. As a result, FucO gene as a furfural reductase using NADH in E. coli is found in [Wang, X., E. Miller, et al., 2011, “Increased furfural tolerance due to overexpression of NADH-dependent oxidoreductase FucO in Escherichia coli strains engineered for the production of ethanol and lactate.” Applied and environmental microbiology 77(15): 5132-5140]. And, in order to find similar genes with the gene in Corynebacterium glutamicum, homology search is conducted using Blastx (http://blast.ncbi.nlm.nih.gov/Blast.cgi). The searching process is schematically shown in FIG. 3.

As a result, among the genes, which are expected to have similarity, cg1310 (SEQ ID No.: 2) showing increased expression at microarray is selected as the target gene. And, information about the selected target gene, and expression level of the target gene depending on the furfural concentration in the medium, which is obtained by analyzing gene expression pattern thereof depending on the furfural concentration using microarray, are shown in the following Table 1.

TABLE 1

| | | | Gene Expression Level | | |
|---|---|---|---|---|---|
| | | | Furfural | Furfural | Furfural |
| | | | Conc. in | Conc. in | Conc. in |
| Gene | Gene | | Medium: | Medium: | Medium: |
| No. | Name | Annotated Protein | 6.5 mM | 13 mM | 20 mM |
| cg1310 | rolM | Maleylacetate | 1.476 | 2.872 | 2.183 |
| | | reductase | | | |

The gene expression level of the above Table 1 shows the relative mRNA expression level of the target gene cg1310 of the wild type strain, Corynebacterium glutamicum ATCC 13032, grown in the medium containing the furfural, against the wild type strain grown without stress in the medium not containing the furfural. As a result of analysis, compared with the medium not containing the furfural, the mRNA expression level of the target gene is increased in the medium containing the furfural, and the mRNA expression level of the target gene is further increased when the furfural concentration is increased from 6.5 mM to 13 mM.

Method Using Gene Expression Analysis

DNA Microarray is used as a method for analyzing gene expression depending on the furfural stress.

Specifically, among genes corresponding to the module classified at http://www.coryneregnet.de/ of entire genes, and genes, whose corresponding protein names contain at least one of transport, permease and pump, genes showing increased expression level at all furfural concentrations are selected, and then, genes showing about 10 folds or more increased expression level at least one concentration are selected. And, among the selected genes, existing genes, which are known to response to stress, are excluded by literature search, and cg0310 (SEQ ID No.: 3), cg1661 (SEQ ID No.: 1), cg3374 (SEQ ID No.: 4) and cg3399 (SEQ ID No.: 5) are selected as the target gene. These are catalase, efflux pump in the cell membrane, oxidoreductase and permease-type genes. Then, information of the target genes selected by microarray, and expression levels of the target genes depending on the furfural concentration in the medium, which are obtained by analyzing gene expression pattern thereof using microarray, are shown in the following Table 2.

TABLE 2

| | | | Gene Expression Level | | |
|---|---|---|---|---|---|
| | | | Furfural | Furfural | Furfural |
| | | | Conc. in | Conc. in | Conc. in |
| Gene | Gene | | Medium: | Medium: | Medium: |
| No. | Name | Annotated Protein | 6.5 mM | 13 mM | 20 mM |
| cg0310 | katA | catalase | 3.219 | 3.443 | 10.956 |
| cg1661 | — | Arsenite efflux | 7.240 | 3.071 | 13.186 |
| | | pump ACR3 or | | | |
| | | related permease | | | |
| cg3374 | cye1 | Putative NADH- | 1.461 | 3.219 | 29.016 |
| | | dependent flavin | | | |
| | | oxidoreductase | | | |
| cg3399 | — | Permease of the | 4.485 | 6.529 | 15.694 |
| | | major facilitator | | | |
| | | superfamily | | | |

The gene expression levels of the above Table 2 show the relative mRNA expression levels of the target genes of the wild type strain grown in the medium containing the furfural, against the wild type strain grown without stress in the medium not containing the furfural. As a result of analysis, compared with the medium not containing the furfural, the mRNA expression levels of each target gene are increased in the medium containing the furfural, and the mRNA expression levels of the target genes are further increased as the furfural concentration is increased.

### Example 3

### Confirm of Resistant Effect of Target Gens in Furfural-Containing Medium

A test for confirming resistant effect of the target genes selected in Example 2 is conducted. At this time, among the target genes of Example 2, cg3399 is excluded from this test because it is not grown after culturing in a medium containing 20 mM furfural.

Specifically, as the target genes of the present disclosure, cg0310, cg1310, cg1661 and cg3374, and b2799 (ACCESSION NP_{—}417279, VERSION NP_{—}417279.2 GI:345452723), which is FucO gene of E. coli K-12 MG1655 (L-1,2-propanediol oxidoreductase), as a positive control group are transformed into a wild type strain, Corynebacterium glutamicum ATCC 13032, using a Corynebacterium glutamicum expression vector, pAN6, under the conditions of 25 μF, 200Ω and 2,500 V by electroporation, respectively. As a negative control group, pAN6, which is not inserted with any other genes, is also transformed into a wild type strain with the same method. The pAN6 (Kan′) is a high copy plasmid, and a shuttle vector derived from pEKEx2 for gene expression regulation of Corynebacterium glutamicum/E. coli (P_{tac}, lacI^{q}, pBL1 oriV_{C.glutamicum}, pUC18 oriV_{E.coli}). This may be confirmed in [Frunzke, J., Engels, V., Hasenbein, S., Gätgens, C., Bott, M., 2008. Co-ordinated regulation of gluconate catabolism and glucose uptake in Corynebacterium glutamicum by two functionally equivalent transcriptional regulators, GntR1 and GntR2. Molecular microbiology. 67, 305-322], and the contents of the literature in its entirety are herein incorporated by reference. After the transformation, cells are selected on BHIS (37 g/L brain heart infusion (Difco), 91 g/L sorbitol) agar plate containing 25 μg/ml Kanamycin. As a result, recombinant strains pAN6-cg0310, pAN6-cg1310, pAN6-cg1661, pAN6-cg3374 and pAN6-b2799 are manufactured, respectively.

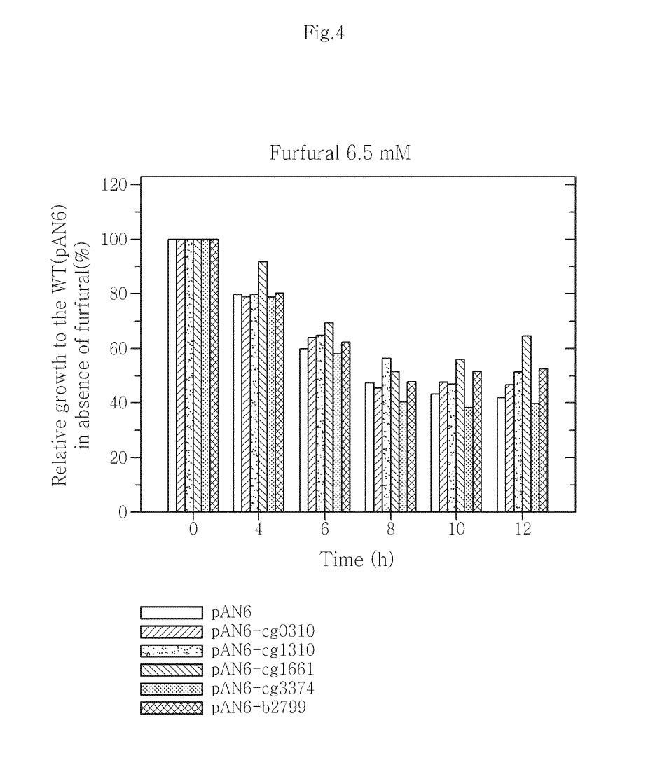

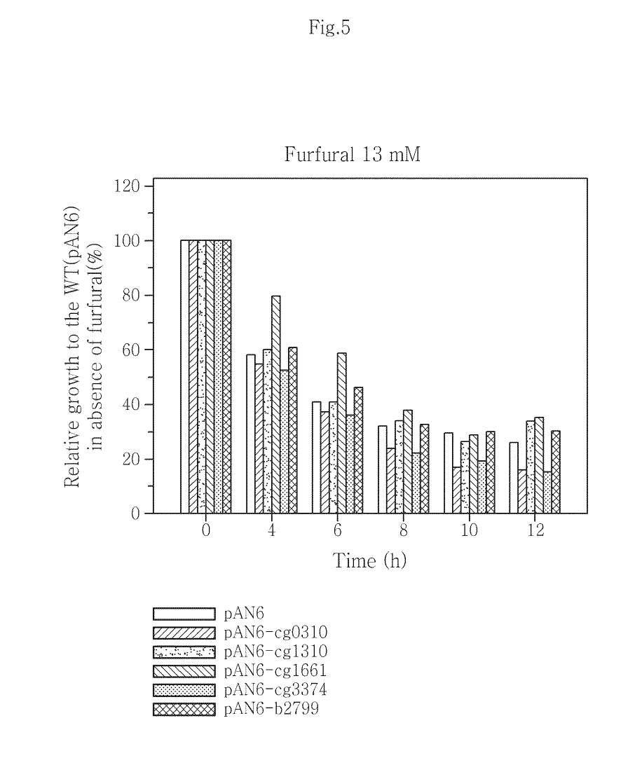

The above pAN6 contains lacI gene. Accordingly, gene expression may be controlled by IPTG induction. Thus, in order to confirm the furfural resistance of the recombinant strains, IPTG is added to the culture at the concentration of 0.5 mM at the time of inoculating the strains for induction, thereby overexpressing the genes. Then, the recombinant strains are cultured in CGXII medium supplemented with 6.5 mM and 13 mM furfural, respectively. And, OD₆₀₀ value by the time is compared with OD₆₀₀ value of the negative control group in the medium not containing the furfural, and the results are shown in FIG. 4 and FIG. 5.

As a result, it is found that the pAN6-cg1310 and pAN6-cg1661 have the highest furfural resistance because they show about 1.5 folds and about 1.8 folds higher cell growth than the negative control group (pAN6) in the medium not containing the furfural. Accordingly, these are decided as the final furfural-resistant genes.

[Accession No.]
Depositary Authority: Korean Collection for Type Culture (Republic of Korea)
Accession No.: KCTC 12565BP
Date of Deposit: 2014 Mar. 5.

While the exemplary embodiments have been shown and described, it will be understood by those skilled in the art that various changes in form and details may be made thereto without departing from the spirit and scope of the present disclosure as defined by the appended claims.

What is claimed is:

1. A furfural-resistant gene comprising at least one genetic sequence selected from the group consisting of cg1661 (SEQ ID No.: 1) and cg1310 (SEQ ID No.: 2) gene.

2. A furfural-resistant strain comprising the gene according to claim 1.

3. The furfural-resistant strain according to claim 2, which is grown in a furfural-containing medium.

4. The furfural-resistant strain according to claim 2, whose growth in the furfural-containing medium is increased about 1.4 folds or more, compared to a wild type wherein the wild type is Corynebacterium glutamicum wild type.

5. The furfural-resistant strain according to claim 4, wherein the wild type is Corynebacterium glutamicum ATCC 13032.

6. The furfural-resistant strain according to claim 5, which is Corynebacterium glutamicum ATCC 13032/pAN6-cg1661 (KCTC 12565BP).

7. A method for producing amino acids, which comprises inoculating the furfural-resistant strain according to claim 2 to a hydrolysate derived from lignocellulosic biomass.

8. A method for screening a furfural-resistant gene, which comprises:
selecting a target gene for identifying the furfural-resistant gene; and
after inserting the target gene into a wild type and overexpressing thereof, selecting a gene showing furfural resistance as the furfural-resistant gene.

9. The method for screening a furfural-resistant gene according to claim 8, wherein said selecting a target gene for identifying the furfural-resistant gene comprises at least one of:
searching genes, which are expected to have furfural-resistance through literature search, and selecting a gene, which is similar with the gene searched through the literature, among genes of the wild type, as the target gene; and
analyzing gene expression pattern depending on furfural stress using microarray, and selecting a gene showing high expression level as the target gene.


SEQUENCE LISTING (5 sequences)

SEQ ID NO: 1 - DNA, 999 bp, Artificial Sequence
  cg1661 which is a furfural-resistant gene
ttgaaacgggtcgttgaagcgtgggatagatttcaaatcccactgtatatcacggctcttatcgccggagcactggtgggtttgcagtggccaggctctacagggggtttcgaaagcgccattaacccagcgttaatggcgctgctttatgccacttttctcggtattccgatcactcggattggtgcagcgctgaaagatctgagatttctcatagtgctcatgtccgtcaattttgttgcagtgcctctggtggcttttgcgttgagcagattcattgcgggtgatgaggcgcttctaatcggatttttactggtgattctcgcgccgtgcattgattacgtcattgtctttgctggtttggccagggccgcccaagacaagctccttgccgccacgccaatattaatgcttgtccaaatcctgctgatccccgtcttcctggctgtttttgtgggttcagatgcccttggctcaatctctttcggcccatttgtagaagcatttttcctcctgattctcattccacttgttgctgctgcgggaactcagcaagtggcaagaaagtggcaggtaggacgtacaattatggctgctgcagaagcaatcatggtgcctttaatgatgctgacgttgttcgctgtcatcgcatcgcaagtggaagctgtgagtggtcaattcaccgatatcgccacagtagtgccactatatgtcgcctttttgatggtgatgattccaattggtggcgggatatccaaactcggtggcttaggtttcaaagagcaacgagccatcgtttttagcggagcaacccgtaactctttggtcgttttacctttagcgttagcacttcccgcaggcctggaaatagcggccgtcgtagttgtcactcaaaccctcgtggaactgattggcatggttgtctacgtgcgcatcatccctttaattttccatgaaaagcagacatacaggaaactttcaggcataggggagtcatga

SEQ ID NO: 2 - DNA, 1071 bp, Artificial Sequence
  cg1310 which is a furfural-resistant gene
atgtctttacagttcgatcatgaaaccctcggtcaacgagttctgttcggttcaggtgaggcggcgcaaaatctcgccgctgaaattagccgactcgatgccaaaaacgtcatggtggttgccggtgatttcgagcttcccatggcacggcaagtagcagcagatattgatgtcaaggtgtggcattcaaatgtcgtgatgcatgtgcccatcgaaacagcagaagaagcacgcagtgttgcgaaagaaaacgacattgatgttgtggtgtgtgtgggcggtggatccacaacaggtctagctaaagcgattgccatgaccaccgcattgccgatcattgcggtacccactacttatgcaggttctgaagcaacaaatgtgtggggattgaccgaagccgcgcgcaaaacaactggtgttgataacaaagtgctgccagtgacagttatctacgattcagcgttaaccatgtctttgccggtagaaatgtcggttgcttctggtctcaatggtttggctcactgcattgattctttgtggggaccgaaggcggatcccatcaatgcggctatggctgctgagggaattcgagcactttctgctggccttcccaagattgtggcagatgctcaggacgtagatggtcgcgatgaagcgctctacggtgcctacctggctgcggtgtcttttgcctctgctggctctggtctccaccacaagatctgccacgtgttgggtggaacttttaaccttccacacgcgcaaacccatgcaacagtactgccttatgttcttgccttcaacgcgccatatgcgccacaggcagaacaacgcgcagcggcagctttcggttctgcgacagcacttgaaggattgcaacagctgcgtgcccaagtgggagcaccacagcgactatccgattacggattcaccgcagcaggaatcccagaggcagtggaaatcatcttggagaaagtaccggcgaataatccacggacggtcacagaagaaaacctcactgcgctgcttaccacagcgctcaacggcgacgatccagcaactttgaattaa

SEQ ID NO: 3 - DNA, 1551 bp, Artificial Sequence
  cg0310 which is KatA expressing Katalase
atgtctgagaagtcagcagcagaccagatcgtagatcgcggaatgcgtccaaagctttctggaaacactacccgccacaacggagcaccagttccatctgagaacatctccgcaaccgcaggcccacagggtccaaacgttctcaatgacattcacctcattgaaaagctcgcacactttaaccgcgagaacgttccagagcgtatccctcacgcaaagggccacggcgctttcggtgagctgcacatcaccgaggacgtatccgaatacaccaaggcagacctgttccagcctggtaaggtcaccccgctggctgttcgcttctctactgttgctggtgagcagggctccccagatacctggcgcgacgtccacggcttcgctcttcgcttctacaccgaagagggcaactacgacatcgtgggtaacaacaccccaaccttcttccttcgtgacggcatgaagttcccggacttcatccactcacagaagcgtctcaacaagaacggtctgcgcgatgcagacatgcagtgggatttctggacccgcgcacctgaatctgcacaccaggtgacctacctgatgggtgaccgcggtacccctaagacctcccgccaccaggacggcttcggctcccacaccttccagtggatcaacgctgaaggtaagccagtttgggttaagtaccacttcaagacccgccagggctgggattgcttcaccgatgcagaagcagcaaaggttgcaggcgagaacgctgactaccagcgcgaagacctctacaacgctattgaaaacggcgacttcccaatctgggacgtcaaggttcagatcatgcctttcgaggatgcagagaactaccgctggaacccattcgacctgaccaagacctggtcccagaaggattacccactgatcccagtcggttacttcatcctgaaccgcaacccacgcaacttcttcgctcagatcgagcagcttgcactggatccaggcaacatcgttcctggcgtcggcctgtccccagaccgcatgctccaggcacgtatcttcgcatacgctgaccagcagcgttaccgcatcggcgctaactaccgcgacctgccagtgaaccgtccaatcaacgaggtcaacacctacagccgcgaaggttccatgcagtacatcttcgacgctgagggcgagccttcctacagccctaaccgctacgacaagggcgcaggctacctggataacggtacggattcctcctccaaccacacctcctacggccaggctgatgacatctacgtcaacccagacccacacggcaccgacctggttcgtgctgcttacgtcaagcaccaggatgatgacgacttcatccagccaggcatcctataccgcgaggtcctggatgagggcgagaaggagcgattggcagacaacatctccaacgcaatgcagggcatctctgaggcaaccgagccacgcgtctacgactactggaacaacgttgatgagaacctcggcgctcgcgtcaaggagctttacctccagaagaaggcttaa

SEQ ID NO: 4 - DNA, 1083 bp, Artificial Sequence
  cg3374 which is Cye1 expressing Putative NADH-dependent flavin oxidoreductase
gtgtccaagctgtttaccccaattcaaatccgcgacatcaccatccccaaccgcgtgtggatgtcaccgatgtgcacctactctgcagccaccggttcaggtcttcccaccgattttcaccaggctcattacgcagctcgcgcagcaggtggtgtcggattagtcatggttgaagcaactggagtgaaccccgtagctcccatctccccagtcgaccttggactttggagccatgaccaaattgaaccattctcccgagtgacagcagctattcgcgccggtggggcagtaccggccgttcaattagcccatgctggccgcaaggcatccaccgatgctccgtggaatggtggcggatatgttggaccagaaaccaatggatgggagactgtcggccccagccctctggcattcccaggtttgcctgctccgcgcgagctgacggtttcagaaatccaagaggttgtgcagcagttcgctggcgccgccgttcgtgccgatcaggctggttttgatgtcgtggaaattcacgcagcacacggctaccttttgcataacttcctttctccgatctccaacaagcgcaccgattcatacggcggatctttagaaaaccgcgctcgcatcgtgctcgaagtcattgatgcaatccgcgcagtgtggccagaggaaaagcctgtattcatgcgcatttccaccaccgactgggtggaggaaaacccacaggatgatcgcgagtcctggacgctgagccaaagcaggcagctggctttgtgggcatccgagcacggagttgatttgatcgatgcctcttctggtggcctcgacatcgtccccattccgcatgaccgcgattaccaaaccgcgaaggccgcagatcttcacgcaagtaccggagtgacagtcgctgctgtggggcgcattgatgacgcccaaactgcgcacaatttggttgattctggcgatgtcaatgcagttttcctcggccgtccactgctcaaggatccttcctgggcaaaccaagcagccctcgcactaggtgcggaacccaggtatgttcaccaatacgactacgtactttaa

SEQ ID NO: 5 - DNA, 510 bp, Artificial Sequence
  cg3399 which is a gene expressing Permease of the major facilitator superfamily
atggccgtttttgatttccctaaccccgttaacgaatatgcagctcgctgcactgcgggtttggttgttttgctgagcgccgccacgctttttgcctctggtgaccttcgtattattttggcaagcatcttgaccttcggcttcgcattgcgcgtcgccggcggaccccgttactccccctttggacgtctttcagtacacgtacttgtaccactgctgaagaaagcaccgatcctgacccctggcccaccaaagcgcttcgcgcagaccatcggcctgggctttagcggtacttcccttatccttatggcctttggatttaacgttgcagcttccgtagtcctagtcatgctcatcgcagcagccaccttagaatccgtctttggtatctgcctcggttgctggggattcggcaagctcatgcgctacggcgtcatcccagaagacgtttgcgagcagtgcttccagaaggaatcctcccgcaccggctggctcgttagcctgaagtag